(19) United States
(12) Patent Application Publication
(10) Pub. No.: US 2002/0129389 A1
(43) Pub. Date: Sep. 12, 2002

(54) Method for determining the in vivo function of DNA coding sequences

(76) Inventors: Patricia L. Kilian, Lidingo (SE); Jerome I. Rotter, Los Angeles, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/096,298

(22) Filed: Mar. 8, 2002

Related U.S. Application Data
(63) Continuation of application No. 09/148,384, filed on Sep. 4, 1998.
(60) Provisional application No. 60/058,165, filed on Sep. 8, 1997.

(51) Int. Cl. A01K 67/00; C12Q 1/68

(52) U.S. Cl. 800/8; 435/6

(57) ABSTRACT

A method for screening a large number of full or partial cDNA coding sequences to determine which are expressed in a correlated manner is disclosed, as well as a method for determining which coding sequences are responsible for the appearance of a phenotypic trait. Additionally, a method for determining the chromosomal locus controlling the expression of a coding sequence responsible for the appearance of a phenotypic trait is disclosed. Also, disclosed is a method for determining the sequential order of a genetic network responsible for the appearance of a phenotypic trait.

This application is a continuation application of U.S. application Ser. No. 09/148,384, filed Sep. 4, 1998, which claims priority to U.S. Provisional Application Serial No. 60/058,165, filed Sep. 8, 1997; both of which are incorporated herein by reference.

### FIELD OF THE INVENTION

The invention is in the field of genomics, specifically, determining the biological role of genes corresponding to full or partial gene sequences.

### BACKGROUND

There are estimated to be between 100,000-150,000 DNA sequences in the human genome which code for specific proteins. The large scale sequencing of human cDNA libraries by the Human Genome Project and commercial-based projects has resulted in the generation of partial gene sequences or Expressed Sequence Tags (ESTs). ESTs are unique DNA sequences approximately 300-400 nucleotides long—sufficient to unequivocably identify a gene. Private and publicly available databases have been generated which contain full or partial sequence information for many or possibly all human genes.

The determination of the function of the identified full and partial length genes represents the most important and most difficult challenge facing the Human Genome Project and commercial large-scale sequencing efforts. There is a particularly urgent need to identify genes and gene networks responsible for many human diseases. The function of approximately half of the genes identified to date remains unknown (S. Oliver, Nature 379: 597-600 (1996)). Current methodologies of determining in vivo function of gene sequences include positional cloning, creation of libraries of knock-out mice, and gene expression using human tissues. These methodologies are slow and inefficient, primarily because they analyze gene sequences one at a time. A rapid, high-throughput method of determining the biological function of gene sequences is needed, particularly those playing a role in human disease.

One currently available methodology for elucidating gene function is positional cloning. Positional cloning involves the isolation of a gene solely on the basis of its chromosomal location, without regard to its biochemical function. The positional cloning approach based on human material has been successfully applied to the identification of genes responsible for single gene diseases, but has not yet been successfully applied to the more common human genetic disorders which involve the interaction of multiple genes such as type II diabetes, obesity, osteoporosis, and inflammatory based disorders. Such complex multigenic diseases involve genes linked in a common genetic network or pathway. Because positional cloning analyzes gene one at a time, it does not allow the identification of downstream drug targets for complex, multigenic diseases.

Another current methodology for identifying gene function is gene expression databases based on human tissues. In this method, the types of tissues expressing a gene as well as its differential expression in normal vs. disease tissue is explored and provides some insight into the pathological role of the gene. However, there are problems associated with using human tissues to study disease. First, major organs and tissues are not readily available except as autopsy material, which is of questionable value for gene expression studies. Second, because the cause of a particular disease may vary widely among unrelated individuals, comparisons of results from unrelated individuals is difficult. The genotype and phenotype of the individual from which the sample is obtained is generally not well known and, thus, the interpretation of results is complicated because environmental effects cannot be readily separated from genetic effects.

Other approaches for determining gene function are based on the creation of knock-out or transgenic mouse models. For instance, Lexicon Genetics, Inc. has developed a method of inactivating or deleting individual ESTs or genes from mice on a genome-wide basis. Their technology is referred to as “Retrovirus Promoter Trap Vectors”—a positive-negative selection which is used in gene targeting experiments in mouse embryonic stem cells. The company is building a library of 500,000 mutant embryonal stem cell lines called OmniBank®, which will be catalogued by the DNA sequence of the particular mutated gene. Accordingly, a customer interested in the phenotypic role of a particular gene would have the mouse line generated from the particular stored embryonal stem cells. There are several limitations to this approach. First, the total elimination of a gene's function is generally not representative of the pathology of most human genetic diseases, which are due to far more subtle changes in gene activity. Second, approximately ⅓ of mouse knock-outs are lethal at the embryonic or neonatal stage and are, thus, uninformative. Third, knocking out a gene's function may cause compensatory development pathways to develop, resulting in an alteration of gene function and phenotype in the adult animal and confounding interpretations of gene function in the “normal” setting. Fourth, the knock-out approach only allows the analysis of one gene at a time. Fifth, it takes a minimum of 10 months to create a knock-out mouse, and it often does not display any phenotypic abnormality.

There are other methods of creating knock-out models. Hexagen, Inc. is using chemical mutagenesis to create knock-out mice, in contrast to retroviral based approaches. Chemical mutagenesis results in the truncation or deletion of one or two genes in an individual animal. A new technology described by Hicks et al. in the August, 1997 issue of Nature Genetics, Volume 16(4), uses a gene trap retrovirus shuttle vector to disrupt genes expressed in murine embryonic stem cells. The authors state that the procedure can be applied to the 10,000-20,000 genes expressed in embryonic stem cells. Thus, this approach is limited to examining only those genes expressed during embryonic development.

Regardless of the method of creating the knock-out model, the drawbacks are the same—only one or two genes can be examined at a time, and the complete elimination of gene function is typically not representative of common human genetic diseases which are due to far more subtle changes in gene activity.

A method for creating transgenic mice with inducible (liver) gene expression in the adult animal has been described in Nature Biotechnology 15:239-243 (1997). The authors state that this approach circumvents the deleterious effect of constitutive gene expression typical of other transgenic over-expression methodologies. However, this method is slow and technically difficult.

Systematic Quantitative Trait Locus (QTL) analysis is a powerful method for determining the chromosomal loci controlling the appearance of phenotypic traits. Traditional QTL analysis was first described by Sax in 1923. It involved comparing the phenotypic means for two classes of progeny: those with marker genotype AB, and those with marker genotype AA. The difference between the means provided an estimate of the phenotypic effect of substituting a B allele for an A allele. Systematic QTL expands upon traditional QTL analysis by employing a whole genome search of genetic markers, known as interval mapping, using detailed maps of genetic markers called restriction fragment length polymorphisms (RFLPs). These RFLPs are spaced, on average, every 100 base pairs in a typical genome. Interval mapping uses phenotypic and genetic marker information to estimate the probable genotype and the most likely QTL effect at every point in the genome, by means of a maximum-likelihood linkage analysis. This pioneering method was first described by E. S. Lander and D. Botstein in Genetics 121:174-199 (1989), and is also described in International Application WO 90/04651. Basically, the methodology for systematically mapping QTLs involves arranging a cross between two inbred strains differing in a phenotypic trait of interest or whose resultant F2 or N2 progeny differ in a phenotypic trait of interest. Segregating progeny are scored both for the trait and for a number of genetic markers. Typically, the segregating progeny are produced by a N2 backcross (F1×Parent) or an F2 intercross (F1×F1). A correlation among the segregating progeny between the appearance of a quantified phenotypic trait and the presence of a genetic marker indicates that the chromosomal loci containing the marker controls the appearance of the phenotypic trait. A computer program called MAPMAKER has been developed to aid in QTL analysis (E. Lander Genomics 1:174-181 (1987)).

Systematic QTL analysis between mouse strains has been used to map the chromosomal locations of genes linked with single gene as well as complex multigene traits. See, e.g., E. Lander and D. Botstein, supra. However, the identification of the genes residing in these QTL regions which are conclusively responsible for a particular phenotype has been accomplished in only a few cases. Also, the gene residing in the QTL region may not be the optimal target for drug discovery or disease diagnosis. Instead, genes or targets lying downstream in the metabolic or other pathway may represent the optimal target.

Systematic QTL analysis was taken one step further in a study by Machleder et al. J. Clin. Invest. 99(6):1406-1419 (1997). In this study, the authors mapped chromosomal loci controlling the transcription of mRNA corresponding to a gene sequence of interest. The authors mapped the genetic factors contributing to the correlation between high density lipoprotein (HDL) levels and atherogenesis in response to diet. They studied mice derived from an intercross between a strains of mice susceptible to atherogenesis—C57BI/6J (B6) and a strain resistant to atherogenesis—C3H/HeJ (C3H) using a complete linkage map/QTL approach. The authors first determined that three distinct genetic loci, on chromosomes 3, 5 and 11, exhibited evidence of linkage to a decrease in HDL-cholesterol after a high fat diet. Next, since cholic acid is required for the diet induced changes in HDL levels and for the development of atherogenesis in these strains, the authors then used the complete linkage map/QTL approach to examine the expression of the enzyme cholesterol-7-alpha hydroxylase (C7AH) in the intercross mice. Expression of C7AH was quantified by measuring the amount of mRNA in liver which hybridized to a cDNA probe. They found that multiple genetic loci contributed to the regulation of C7AH mRNA levels in response to a high fat diet, the most notable of which coincided with the loci on chromosomes 3, 5 and 11 previously linked to a decrease in HDL-cholesterol levels after a high fat diet.

### BRIEF SUMMARY OF THE INVENTION

The present invention is directed to a method for screening one or more Expressed Sequence Tags (ESTs) for in vivo function and possible therapeutic relevance.

According to the first aspect of the invention, a large number of partial or full length gene sequences, hereinafter referred to collectively as “coding sequences”, can be examined simultaneously to determine which, if any, are expressed in a correlated manner. Specifically, the amount of transcribed mRNA corresponding to each examined coding sequence is measured in cells, tissues, organs, blood and other samples obtained from a genetically diverse population of organisms, preferably animals, and most optimally mice, to give an expression profile for each coding sequence examined. Expression profile is defined to be the level of transcribed mRNA from a selected tissue which corresponds to a particular coding sequence of interest. If the expression profile of any one coding sequence correlates either positively or negatively with an expression profile of one or more of the other coding sequences, these coding sequences are deemed to be linked in a common genetic network or pathway.

According to a second aspect of the invention, the expression profiles of a large number of coding sequences are determined as in the first aspect of the invention, additionally, each progeny are scored for a quantifiable phenotypic trait. In a preferred embodiment, the quantifiable phenotypic trait is a disease state. A correlation between the expression profiles of coding sequences linked in a genetic network and the appearance of a phenotypic trait indicates that the coding sequences in the genetic network determine the appearance of the phenotypic trait.

According to a third aspect of the invention, the expression profiles of a large number of coding sequences are determined as in the first or second aspects of the invention, additionally, genotypic profiles of each of the progeny are determined using detailed maps of genetic markers covering the entire genome of the organisms. A correlation between the expression profile of a coding sequence linked in a genetic network and a specific marker region indicates that the marker region controls the expression of that coding sequence.

In a fourth aspect of the invention, the expression profiles of a large number of coding sequences are determined and correlated with the genotypic and phenotypic profiles of each of the progeny, additionally, the coding sequences linked in a common genetic network are hybridized to the chromosomal DNA. The sequential genetic pathway can be then determined depending on whether the coding sequence hybridizes to the same chromosomal loci controlling the expression of that coding sequence.

### DETAILED DESCRIPTION

The invention relates to a rapid and high throughput method for determining the in vivo function and therapeutic relevance of partial or complete gene sequences, referred to hereinafter as “coding sequences”. Current methodologies are slow and require examining coding sequences one at a time. With the method of the present invention, the expression profiles of a large number of coding sequences can be determined simultaneously and (I) correlated with each other to determine a common genetic network or pathway; (II) correlated with each other and with the appearance of a quantifiable phenotypic trait to determine whether the common genetic network controls the appearance of the phenotypic trait; (III) correlated with the genotypic profile of the progeny to determine the chromosomal loci controlling the expression of the coding sequences; and (IV) correlated with the genotypic and phenotypic profiles of the progeny and the chromosomal loci to which the coding sequences hybridize to determine the sequential order of genes in a genetic network responsible for a phenotypic trait.

The first step of the method of the invention is to generate a large number of animals with extensive genetic diversity. Although the method of the present invention can be used to examine coding sequences from any organism, in a preferred embodiment, human coding sequences are examined. In order to profile the expression of human coding sequences, the type of animal selected should have a high degree of gene sequence conservation with humans. Mouse and human gene sequences are strongly conserved, and their small size and ease of care make mice the preferable animal model of human gene expression.

The mouse is a powerful model for the study of human biology and pathology. There are numerous studies showing the relevance of mouse models to the study of human disease. Mouse and human gene sequences are strongly conserved. The average degree of nucleotide sequence identity between mouse and human expressed sequences is approximately 85% (Makalowski et al. Genome Research 6:846-57 (1996)). Thus, the function of human gene sequences can be productively investigated in mouse models. Animal studies should identify key genes acting in the same biochemical pathway or physiological system as humans.

A group of animals with extensive, yet identifiable, genetic diversity is generated by performing two sets of crosses with two highly inbred progenitor strains. The resulting group of animals is referred to as the intercross, or F2 generation. Alternatively, members of the F1 generation can be backcrossed with the parental strain producing an N2 generation. The progenitor strains are selected on the basis of the phenotypic trait or therapeutic area of interest. Thus, for example, the C3H/HeJ and B6 strains of mice can serve as progenitor strains for studies on vascular lesions and atherosclerosis because they differ greatly in their susceptibility to lesions on a high fat diet. The offspring from the initial cross, the F1 animals, inherit one copy of each chromosome from one parent, in this example, C3H/HeJ, and a second copy from the other parent, in this example, B6. Thus, each animal in the F1 generation is genotypically identical (all heterozygous) and phenotypically identical.

The F1 hybrid animals are then bred with each other to produce a large set of F2 animals (for example, 200-1000 animals), or can be bred with the parental strain producing an N2 backcross generation. If an F2 intercross is performed, each F2 animal will have a unique genotype because of the segregation of progenitor alleles from the heterozygous F1 animals. Some loci will be homozygous for one of the progenitor alleles, some will be homozygous for the other progenitor allele, and some will be heterozygous with both alleles.

Alternatively, the F1 hybrid animals may be backcrossed with one of the progenitor strains (e.g., B6). In this case, the so-called N2 animals will be either homozygous (e.g., both alleles are from the B6 progenitor) or heterozygous (e.g., one allele from B6 and the other from C3H/HeJ).

The F2 or N2 animals are then subjected to an experimental regimen under controlled conditions. Experimental regimen is defined to include any environmental condition or pressure imposed equally on all the F2 or N2 animals. For example, if the therapeutic area of interest is the development of atherosclerosis and an F2 intercross is generated, all of the F2 animals would be put on a high fat diet for a period of time. At the end of this period, each of the F2 animals is phenotyped. For example, blood lipid levels, glucose, insulin, circulating factors, histological exams, body weight (percent and site of deposition), etc. can be measured (see Fisler, et al. Obesity Research 1(4): 271-280 (1993), Warden et al. J. Clin. Invest. 92:773-779 (1993)). Animals are then sacrificed and selected organs and tissues retained for gene expression studies.

The next step of the invention is gene expression profiling. The presence or absence or relative abundance of the mRNA corresponding to any of the ESTs being examined is determined. Selected tissues and organs from each of the F2 animals are individually analyzed. The types of tissues and organs selected for study may vary depending on the therapeutic area of interest or may be representative of each of the major organs (e.g., liver, muscle, fat, pancreas, bone, brain or brain regions, heart). Total mRNA is obtained from each tissue or organ and cDNA may be prepared. Total mRNA can be isolated from selected tissues or organs using commercially available RNA kits, and other method are well known by those skilled in the art, for example, as described in D. Machleder et al. J. Clin. Invest. 99(6):1406-1419 (1997). Methods for preparing cDNA from mRNA are also well known in the art, for example, as described in the book “Fingerprinting Methods Based on Arbitrarily Primed PCR” by M. Michelli and R. Bova, Springer Publishers (1997).

The genes or partial gene coding sequences to be profiled may correspond to ESTs. As stated above, a large number of human coding sequences represented by ESTs are known and possibly represent the entire repertoire of expressed human genes. Some, but not all mouse ESTs are known. If human coding sequences are being examined for possible in vivo function using a mouse model, that is, profiling the expression of mouse genes corresponding to human coding sequences, one would rely on the high degree of homology between human and mouse coding sequences and use the human coding sequences as probes to detect corresponding mouse mRNA.

For example, total mRNA is prepared from the livers of F2 mice. For each F2 mouse, the presence or absence or relative abundance of mRNA corresponding to each of the coding sequences being investigated is determined. A variety of techniques well known in the art can be used to make this determination, including cross-hybridization of the coding sequence with mRNA, or its corresponding cDNA, direct sequence comparison, mass spectrometry techniques, chip technologies and gel based methods.

In the first aspect of the invention, total mRNA from one given tissue or organ is hybridized to coding sequences of interest. Next, the levels of mRNA transcription for each of the coding sequences are correlated with each other. Those coding sequences showing a correlation (either positively or negatively) are linked in a common genetic network or pathway. This can be shown more clearly by example. Table I shows a hypothetical of data generated by determining the amount of mRNA transcription corresponding to five ESTs in five F2 mice progeny. It should be noted that a far larger number of ESTs or coding sequences and a far larger number of animal progeny can be simultaneously analyzed according to the method of the present invention. Please note that levels of transcribed mRNA can be examined in one or multiple tissues or organs.

TABLE I

| | Mouse 1 | Mouse 2 | Mouse 3 | Mouse 4 | Mouse 5 |
|---|---|---|---|---|---|
| EST1 | hi | hi | Mid | lo | lo |
| EST2 | lo | hi | Lo | lo | lo |
| EST3 | mid | mid | Mid | mid | mid |
| EST4 | hi | hi | Mid | lo | lo |
| EST5 | lo | lo | Mid | hi | hi |

As can be seen from the hypothetical data, the transcription of mRNA from ESTs 1, 4 and 5 are correlated. In this example, EST5 expression is inversely correlated with that of EST1 and EST4. This may be true when the expression of different coding sequences is measured in different tissues, for example, EST1 and EST4 expression measured in the liver, while EST5 expression measured in adipose tissue. Hence, mice genes corresponding to these ESTs 1, 4 and 5 are deemed linked by a common genetic network or pathway. No genotyping of the animals is necessary to obtain the above result. It should be noted that mRNA levels may have to be normalized to the mRNA of a gene whose transcription level is known to be constant or well defined, such as that of a housekeeping gene.

In a second aspect of the invention, the expression profiles of several coding sequences are examined for correlation not only with each other, but also with the appearance of a quantifiable phenotypic trait. In a preferred embodiment, the phenotypic trait is a disease state. A hypothetical range of outcomes is represented in Table II where the phenotypic trait under investigation is obesity in mice. Again, it should be noted that a far greater number of mice and coding sequences can be examined with this method, and the coding sequence profiles can be gathered from different tissues.

TABLE II

| | F2-1 | F2-2 | F2-50 | F2-80 | F2-200 |
|---|---|---|---|---|---|
| Phenotype (% | 10% | 11% | 20% | 45% | 46% |
| fat) | | | | | |
| EST1 | mid | mid | Mid | mid | mid |
| EST2 | high | low | Low | mid | low |
| EST3 | high | high | High | high | high |
| EST4 | high | high | High | high | high |
| EST5 | low | low | Mid | high | high |
| EST6 | high | high | Low | low | high |
| EST7 | high | mid | Mid | mid | mid |
| EST8 | low | low | Low | low | low |
| ESTX | mid | High | Mid | high | high |

In this set of data, the level of expression of a mouse gene corresponding to EST5, as measured by the relative amount of transcribed mRNA, correlates with the amount of body fat in the animal. This indicates that the mouse gene corresponding to EST5 is a “disease gene” in that it has some role in obesity or associated events. Please note that ESTs 1-5 are not necessarily the same ESTs presented in Table I.

A third aspect of the invention is a method of determining the chromosomal region or regions controlling the transcription of a disease gene. The first step is to determine the genotype of every F2 animal. This is referred to as the genotypic profile. The genome of every organism contains genetic markers every few hundred base pairs, on average, consisting of dinucleotide repeat sequences. The location and sequences of markers are known for the mouse. These marker regions provide a means of determining whether the specified region of the mouse chromosome is derived from one progenitor strain or the other and whether the specified region is homozygous or heterozygous. To determine F2 animal genotype, DNA is extracted from tail clips from each F2 animal. The DNA is cross hybridized with the genotype markers and amplified. The samples are run on the ABI 377. In addition to using the ABI 377, other methods are well known in the art for performing genotypic analysis. The data are analyzed and the genotype make-up of each animal is determined at every region of the genome. As mentioned in the Background to the Invention, a method of identifying the chromosomal region controlling a quantitative phenotypic trait using RFLP linkage maps was first described by Lander, E. et al. In Genetics 121:185-199 (1989). A detailed description of the method of determining quantitative trait loci using RFLP maps is described in U.S. Pat. No. 5,385,835, issued to Helentjaris et al. on Jan. 31, 1995 entitled: “Identification and Localization and Introgression into Plants of Desired Multigenic Traits”. This patent and all other patent and article references cited in this disclosure are incorporated herein by reference. Additionally, a computer program called MAPMAKER has been developed to aid in QTL analysis (E. Lander, Genomics 1:174-181 (1987)).

The next step in the third aspect of the invention is to determine if any correlation exists between the expression profile of a coding sequence associated with a particular phenotype and the genotypic makeup of particular marker regions. Any correlation indicates that the chromosomal loci defined by the marker region controls expression of the coding sequence, which in turn controls the appearance of the phenotypic trait. Again, this can best be explained by example. Data for a hypothetical example is presented in Table III.

TABLE III

| | F2-1 | F2-2 | F2-50 | F2-80 | F2-200 |
|---|---|---|---|---|---|
| Phenotype (% | 10% | 11% | 20% | 45% | 46% |
| fat) | | | | | |
| Genotype | P2, P2 | P1, P1 | P1, P2 | P2, P2 | P2, P2 |
| marker a | | | | | |
| marker b | P1, P1 | P1, P1 | P1, P2 | P2, P2 | P2, P2 |
| marker c | P2, P1 | P1, P1 | P2, P2 | P2, P2 | P1, P2 |
| marker d | P1, P1 | P1, P1 | P1, P2 | P1, P2 | P2, P1 |
| EST1 | mid | mid | Mid | mid | mid |
| EST2 | high | low | Low | mid | low |
| EST3 | high | high | High | high | high |
| EST4 | high | high | High | high | high |
| EST5 | low | low | Mid | high | high |
| EST6 | high | high | Low | low | high |
| EST7 | high | mid | Mid | mid | mid |
| EST8 | low | low | Low | low | low |
| ESTx | mid | high | Mid | high | high |

Table III expands on Table II by including an additional matrix of marker region genotype information for each of the same F2 animals. Again, this data is only representative of a hypothetical analysis. As many as 100-400 genotypic markers may be analyzed simultaneously, and, of course many coding sequences and many more animal progeny would typically be examined. In this hypothetical example, a mouse gene corresponding to EST5 has already been determined to play a role in obesity. Additionally, the genotypes for marker b indicate that the level of expression of EST5 rises as the marker b genotype changes from homozygous for progenitor strain alleles PI to homozygous for progenitor strain allele P2. This would indicate that the gene corresponding to EST5 exists on the marker b region of the P2 derived allele, and that this gene is responsible for the phenotypic trait percentage body fat.

A fourth aspect of the invention involves determining the specific order of the interaction of genes involved in a multi-genic, complex phenotypic trait. As discussed in the Background section, relatively few genetic diseases are controlled by a single gene. It has been estimated that disorders such as atherosclerosis and asthma involve the interaction of over a hundred individual genes. The method of the fourth aspect of the present invention discloses a way of determining the sequential order of the interaction of multiple genes involved in a multi-genic disorder. The expression profiles of multiple coding sequences are determined as before. This expression profile information is correlated with phenotypic measurements, i.e., the phenotypic profile and genotypic data, i.e., the genotypic profile, as detailed in the third aspect of the invention. Additionally, chromosomal mapping of the coding sequence is performed. This is done by any number of techniques well known in the art, such as fluorescent in situ hybridization (FISH). The final step is to determine if the chromosomal loci already determined by systematic QTL analysis to be controlling the transcription of the coding sequences coincides with the chromosomal region to which the coding sequence maps. For example, let us suppose that the expression profiles of three coding sequences, X, Y and Z have been determined to be associated with a particular disease state, that their QTLs controlling the expression of X, Y and Z have been determined, and that the specific regions along the chromosome to which the cDNA for the transcripts of X, Y and Z have also been determined. There are two possible scenarios. First, the cDNA for coding sequence X maps to the same chromosomal locus as the QTL controlling the expression of X. This would indicate that the protein product of gene X is directly responsible for the appearance of the disease state. Schematically, this could be represented as:

X - - - >appearance of the disease state

A second possible scenario is that the cDNA for coding sequence X maps to the QTL controlling the expression of Y. This would indicate that the protein product of gene X controls the expression of Y. Schematically, this could be represented as:

X - - - >expression of Y

Turning to Y, two scenarios are again possible. First, the cDNA for coding sequence Y maps to the same chromosomal locus as the QTL controlling the expression of Y. If this were the case, it could be represented schematically as:

X - - - >expression of Y - - - >appearance of the disease state

Alternatively, the cDNA for coding sequence Y could map to the QTL controlling the expression of some other coding sequence, say Z. This could be represented schematically as:

X - - - >expression of Y - - - >expression of Z

Turning to Z, the same two possibilities exist, and the analysis can be extended for as many coding sequences as were determined to be associated with the disease state. In this way, the genetic sequence of a genetic network consisting of as many as dozens of genes can be elucidated.

Although the invention has been described with reference to presently preferred embodiments, it should be understood that various modifications can be made without departing from the spirit or scope of the invention.

What is claimed is:

1. A method of determining the sequential order of the interaction of multiple genes involved in a multi-genic disorder comprising:
a) crossing two strains of interest to produce progeny, wherein said two strains of interest have differing phenotypes for an initial phenotypic trait associated with said multi-genic disorder;
b) carrying out two crosses, which are either back-crosses or intercrosses, to produce a large set of N2 or F2 progeny;
c) scoring the N2 or F2 progeny for the amount of transcribed mRNA isolated from the progeny corresponding to each coding sequence of a plurality of coding sequences;
d) scoring the N2 or F2 progeny for one or more phenotypic traits of interest, wherein said large set of N2 or F2 progeny comprises variability in said one or more phenotypic traits of interest, wherein each phenotypic trait of interest is quantifiable;
e) scoring the N2 or F2 progeny for genetic markers, wherein each genetic marker defines a chromosomal locus;
f) identifying one or more coding sequences of interest which correlates with said phenotypic trait of interest by comparing the amount of transcribed mRNA of each coding sequence of said one or more coding sequences of interest with the trend of the quantity of said phenotypic trait of interest for a plurality of the individuals of the N2 or F2 progeny;
g) identifying a genetic marker of said selected genetic markers that correlates with at least one of the one or more coding sequences identified in step f), so that a coding sequence is identified with the chromosomal locus defined by the genetic marker, wherein the presence of the genetic marker correlates with the quantity of said phenotypic trait of interest;
h) mapping the cDNA of each coding sequence of interest of the one or more coding sequences identified to correlate with a genetic marker to a specific chromosomal location;
i) determining whether the chromosomal loci controlling the expression of coding sequences associated with the phenotypic trait as in step g) coincide with the chromosomal location to which the cDNA map as in step h); and
j) determining the sequential order of at least two coding sequences of interest in relation to said phenotypic trait;
wherein said at least two coding sequences map to two different genetic markers.

2. The method according to claim 1, wherein said two strains of interest are two animal strains of interest.

3. The method according to claim 2, wherein said two strains of interest are two mouse strains of interest.

4. The method according to claim 1, wherein said multi-genic disorder is atherosclerosis or obesity.

5. The method according to claim 4, wherein said phenotypic trait of interest is percent fat, blood lipid level, blood glucose level, blood insulin level, body weight, or body fat.

6. The method according to claim 5, further comprising the step:
putting all N2 or F2 progeny on a high fat diet prior to step c), step d), and step e).

7. The method according to claim 1, wherein said plurality of coding sequences is a plurality of human coding sequences.

8. The method according to claim 1, wherein each said phenotypic trait of interest is a disease state.

9. The method according to claim 8, wherein said disease state is related to said multi-genic disorder.

10. The method according to claim 1, wherein said initial phenotypic trait is different from said phenotypic traits of interest.

11. A method of determining the sequential order of the interaction of multiple genes involved in a multi-genic disorder comprising:
a) crossing two strains of interest to produce progeny, wherein said two strains of interest have differing phenotypes for an initial phenotypic trait associated with said multi-genic disorder;
b) carrying out two crosses, which are either back-crosses or intercrosses, to produce a large set of N2 or F2 progeny;
c) measuring the quantifiable phenotypic traits for said N2 or F2 progeny;
d) determining the expression profile of a set of coding sequences for said N2 or F2 progeny;
e) comparing the trend of said quantifiable phenotypic traits and the trend of said coding sequences to determine each quantifiable phenotypic trait that correlates with each coding sequence;
f) determining the genotypic profile of a set of genetic markers for said N2 or F2 progeny;
g) identifying a genetic marker that correlates with said quantifiable phenotypic trait that correlates with said coding sequence identified in step f), so that a coding sequence is identified with the chromosomal locus defined by the genetic marker, wherein the presence of the genetic marker correlates with the quantity of said phenotypic trait of interest;
h) determining the chromosomal location of said coding sequence;
i) determining the chromosomal location of said genetic marker;
j) determining whether the chromosomal location of said coding sequence coincides the chromosomal location of said genetic marker; and
k) determining the sequential order of at least two coding sequences of interest in relation to said phenotypic trait;
wherein said at least two coding sequences map to two distinguishable genetic markers.

12. The method according to claim 10, wherein said two strains of interest are two animal strains of interest.

13. The method according to claim 12, wherein said two strains of interest are two mouse strains of interest.

14. The method according to claim 11, wherein said multi-genic disorder is atherosclerosis or obesity.

15. The method according to claim 14, wherein said phenotypic trait of interest is percent fat, blood lipid level, blood glucose level, blood insulin level, body weight, or body fat.

16. The method according to claim 15, further comprising the step:
putting all N2 or F2 progeny on a high fat diet prior to step c) and step d).

17. The method according to claim 11, wherein said plurality of coding sequences is a plurality of human coding sequences.

18. The method according to claim 11, wherein each said quantifiable phenotypic trait is a disease state.

19. The method according to claim 18, wherein said disease state is related to said multi-genic disorder.

20. The method according to claim 11, wherein said initial phenotypic trait is different from said quantifiable phenotypic trait.
